(19) United States
(12) Patent Application Publication
(10) Pub. No.: US 2004/0153061 A1
(43) Pub. Date: Aug. 5, 2004

(54) Adjustable bipolars

(76) Inventor: Chao-Jan Wang, Hsinchu (TW)

Correspondence Address: Chao-Jan Wang, 4f.-8, No. 123, Sec. 3, Taijunggang Rd., Shituen Chiu, Taichung, TW

(21) Appl. No.: 10/355,137

(22) Filed: Jan. 31, 2003

(51) Int. Cl. A61B 18/18

(52) U.S. Cl. 606/51

(57) ABSTRACT

A bipolar includes two parts and each part has a heating distal end. A wire is connected to each part and electrically connected to the heating distal end corresponding thereto. A connection member is connected to the two parts and includes two terminals which are respectively connected to the two wires. An adjusting device which can be a sleeve movably mounted to the two parts or a pin which pivotably connects the two heating distal ends. By the adjusting device, the available length of the bipolar can be optionally used.

### FIELD OF THE INVENTION

The present invention relates to a bipolar that has an adjusting device to adjust the available length of the bipolar.

### BACKGROUND OF THE INVENTION

A conventional way to stop bleeding is to use bipolar to clamp the capillaries and the bipolar is powered by electric energy so as to produce heat to burn and seal the orifice of the capillaries. The conventional bipolar includes two parts which are connected at a connection member so that a gap is defined between the two parts. The user has to hold the two parts and squeeze the two parts toward each other to let the distal ends of the two parts to clamp the capillaries. An inherent shortcoming of the conventional bipolar is that the two distal ends are often not located at different planes so that the two distal ends cannot be cooperated to clamp the capillaries and this cannot successfully burn and seal the capillaries. Besides, the two parts have fixed length so that the users have pick or switch different sets of the bipolar for dealing with different situations.

The present invention intends to provide a bipolar that has an adjusting device so as to easily adjust the length of the two parts of the bipolar.

### SUMMARY OF THE INVENTION

In accordance with one aspect of the present invention, there is provided a bipolar which comprises a first part and a second part. Each of the two parts has a heating distal end and the two heating parts are pivotably connected with each other by a pin. Each part has a wire connected thereto and the wires are connected to two terminals of a connection member to which the two parts are connected.

The pin can be replaced by a sleeve which is movably mounted to the two parts so that by pivoting the two parts about the pin or moving the sleeve, the available length of the two parts can be adjusted.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show a first embodiment of the bipolar of the present invention;

FIG. 2 is a perspective view to show a second embodiment of the bipolar of the present invention;

FIG. 3 shows the operation of the first embodiment of the bipolar of the present invention, and

FIG. 4 shows the operation of the second embodiment of the bipolar of the present invention.

### DETAILED DESCRIPTION OF THE PREFERRED EMBODIMENT

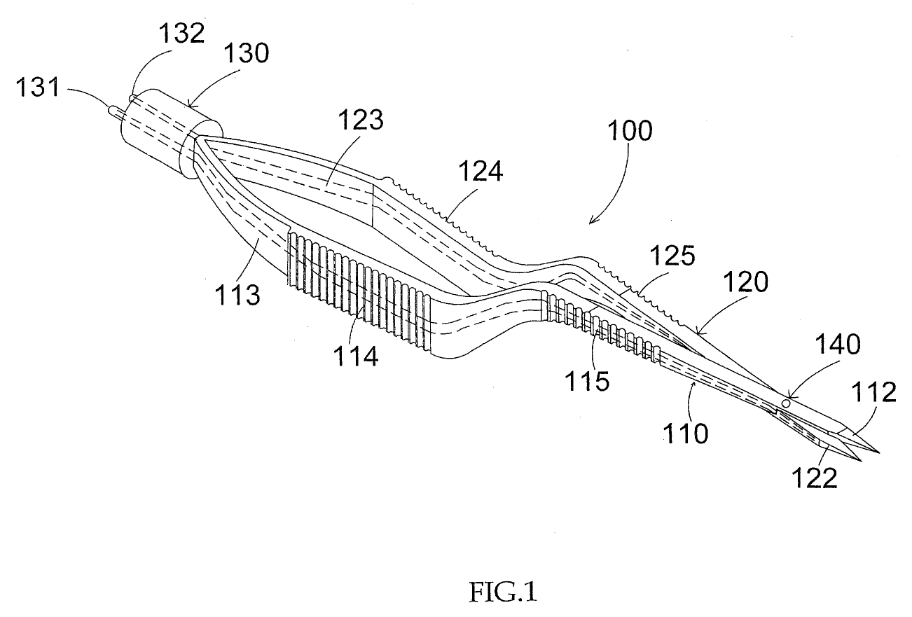

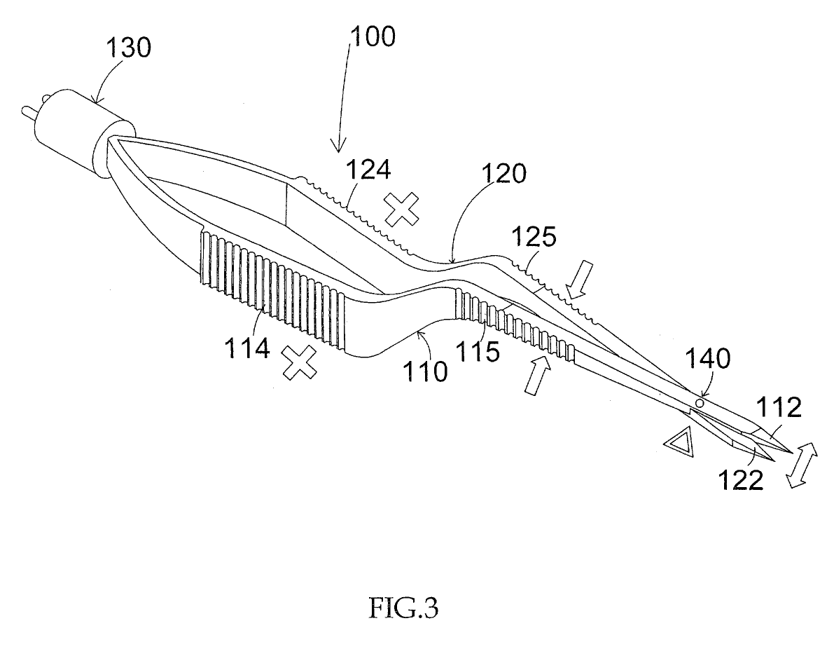

Referring to FIGS. 1 and 3, the bipolar 100 of the present invention comprises a first part 110 which having a first heating distal end 112 at a first end of the first part 110 and a second end which is connected to a connection member 130. A wire 113 is connected to an inside of the first part 110 and electrically connected to the first heating distal end 112. The first part 110 has a holding section 114 and a squeezing section 115.

The second part 120 has a second heating distal end 122 at a first end of the second part 120 and a second end of the second part 120 is connected to the connection member 130. A wire 123 is connected to an inside of the second part 120 and is electrically connected to the second heating distal end 122. Two terminals 131, 132 extend from the connection member 130 and are connected to the two wires 113, 123 of the first part 110 and the second part 120 respectively so that a power source which is not shown can be connected to the two terminals 131, 132 to provide electric energy to the two heating distal ends 112, 122. The second part 120 has a holding section 124 and a squeezing section 112.

The first heating distal end 112 and the second heating distal end 122 is pivotably connected by a pin 141 like scissors so that the first heating distal end 112 and the second heating distal end 122 are pivoted about the pin 140 and moved relative to each other.

The user holds the holding sections 114, 124 and pushes the squeezing sections 115, 125 so as to let the two heating distal ends 112, 122 pivot about the pin 140 to adjust the width therebetween.

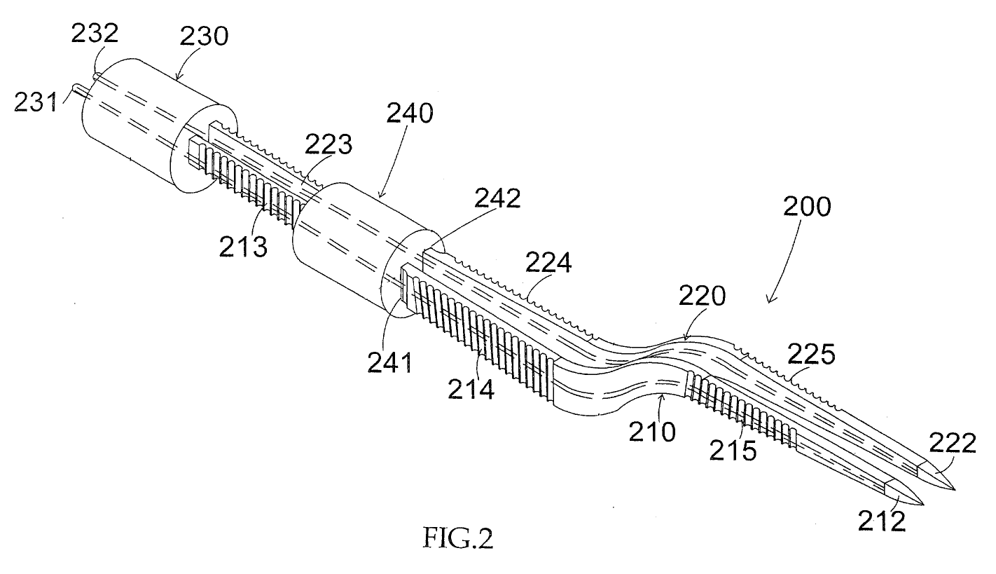

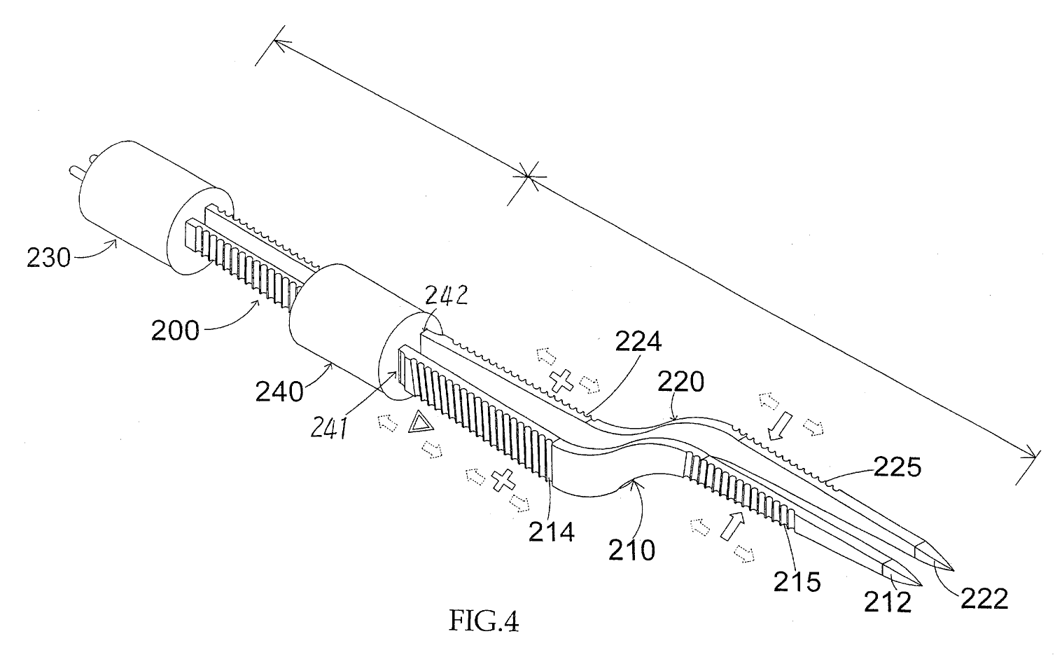

Referring FIGS. 2 and 4, a second embodiment of the bipolar 200 comprises a first part 210 having a first heating distal end 212 at a first end of the first part 210 and a second end of the first part 210 is connected to a connection member 230. A wire 213 is connected to an inside of the first part 210 and electrically connected to the first heating distal end 212 and a terminal 231 of the connection member 230. The first part 210 has a holding section 214 and a squeezing section 215.

A second part 220 has a second heating distal end 222 at a first end of the second part 220 and a second end of the second part 220 is connected to the connection member 230. A wire 223 is connected to an inside of the second part 220 and is electrically connected to the second heating distal end 222 and the other terminal 232 of the connection member 230. A sleeve 240 has two passages 241, 242 through which the first part 210 and the second part 220 movably extend. The second part 220 has a holding section 224 and a squeezing section 225.

By moving the sleeve 240, the length from the sleeve 240 to the heating distal ends 212, 222 can be adjusted.

While we have shown and described the embodiment in accordance with the present invention, it should be clear to those skilled in the art that further embodiments may be made without departing from the scope of the present invention.

What is claimed is:

1. A bipolar comprising:
a first part having a first heating distal end at a first end of the first part, a wire connected to the first part and connected to the first heating distal end;
a second part having a second heating distal end at a first end of the second part, a wire connected to the second part and connected to the second heating distal end, the first heating distal end and the second heating distal end being pivotably connected by a pin so that the first heating distal end and the second heating distal end are pivoted about the pin and moved relative to each other, and
a connection member to which a second end of the first part and a second end of the second part are connected, two terminals extending from the connection member and connected to the two wires of the first part and the second part.

2. A bipolar comprising:
a first part having a first heating distal end at a first end of the first part, a wire connected to the first part and connected to the first heating distal end;
a second part having a second heating distal end at a first end of the second part, a wire connected to the second part and connected to the second heating distal end, a sleeve having two passages and the first part and the second part movably extend through the two passages, and
a connection member to which a second end of the first part and a second end of the second part are connected, two terminals extending from the connection member and connected to the two wires of the first part and the second part.
